(19) United States
(12) Plant Patent Application Publication
(10) Pub. No.: US 2013/0291255 P1
(43) Pub. Date: Oct. 31, 2013

(54) Phalaenopsis orchid plant named 'Mamamia'

(50) ;

(71) Applicant: FLORICULTURA, Heemskerk (NL)

(72) Inventor: René Schoone, Assendelft (NL)

(21) Appl. No.: 13/986,327

(22) Filed: Apr. 22, 2013

Related U.S. Application Data
(60) Provisional application No. 61/637,995, filed on Apr. 25, 2012.

(30) Foreign Application Priority Data
Apr. 26, 2012 (NL) OPS902

(51) Int. Cl. A01H 5/00

(52) U.S. Cl. PLT/311

(57) ABSTRACT

A new and distinct Phalaenopsis plant named ‘Mamamia’ particularly characterized by flowers which are purple/violet with a small white edge; plants which may be propagated economically and uniformly using tissue culture; plants which produce more than one inflorescence; long and sturdy inflorescences; and relatively short, dark-green foliage.

### LATIN NAME OF THE GENUS AND SPECIES OF THE PLANT CLAIMED

Phalaenopsis hybrid

### VARIETY DENOMINATION

‘Mamamia’

### BACKGROUND OF THE INVENTION

The present invention relates to a new and distinct cultivar of Phalaenopsis plant, botanically known as Phalaenopsis of the Orchidaceae family, and hereinafter referred to by the cultivar name ‘Mamamia’.

Phalaenopsis comprises a genus of about 55 species of herbaceous perennials many of which, or the hybrids thereof, are suitable for cultivation in the home or greenhouse. Phalaenopsis is predominantly epiphytic or rock-dwelling, and is native to tropical Asia, the Malay Archipelago, and Oceania. The species typically has 2-ranked, fleshy, oblong or elliptic leaves affixed to a short central stem (monopodial growth), which vary in size from 5 to 8 inches to over 2 feet. The leaves may be entirely green or mottled with silver grey.

Phalaenopsis orchids, often referred to as ‘Moth Orchids’ in the horticultural trade, are frequently used to furnish cut flowers for the florist trade or sold as flowering potted-plants for home or interiorscape.

Phalaenopsis produces upright or pendent lateral racemes, often with many showy flowers which open in succession beginning with the lowermost. The flowers possess three sepals and three petals; the lateral ones being alike. The lowermost petal, called the labellum, is three-lobed and is often more brightly-colored than the other flower segments. Flower colors include various shades of pink, white, yellow and red-brown.

Phalaenopsis orchids are typically propagated from seeds. Asexual propagation of Phalaenopsis is often done from off-shoots which frequently arise from the lower bracts of the inflorescence. The resulting plants are detached from the mother plant and may be planted in a suitable substrate.

The new Phalaenopsis ‘Mamamia’ is a product of a controlled breeding program conducted by the inventor, René Schoone, in Strengweg, Heemskerk, The Netherlands. The objective of the breeding program was to develop a new Phalaenopsis cultivar particularly characterized by its attractive and unique colored flowers, economical propagation via tissue culture, rapid growth, and a plant dimension suitable for packaging and shipping to the market.

The new Phalaenopsis ‘Mamamia’ originated from a cross made by the inventor in 1999 in Strengweg, Heemskerk, The Netherlands. The female or seed parent is the Phalaenopsis cultivar designated ‘Super Stupid’, unpatented. The male or pollen parent is the Phalaenopsis cultivar designated ‘Zuma Pixie’, unpatented. The new Phalaenopsis ‘Mamamia’ was discovered and selected by the inventor as a single flowering plant within the progeny of the stated cross in a controlled environment in 2007 in Strengweg, Heemskerk, The Netherlands.

Asexual reproduction of the new Phalaenopsis cultivar by tissue culture was first performed in November, 2007 in Cieweg 13, Heemskerk, The Netherlands, and has demonstrated that the combination of characteristics as herein disclosed for the new cultivar are firmly fixed and retained through successive generations of asexual reproduction. The new cultivar reproduces true to type.

### BRIEF DESCRIPTION OF THE INVENTION

The following traits have been repeatedly observed and are determined to be unique characteristics of ‘Mamamia’, which in combination distinguish this Phalaenopsis as a new and distinct cultivar:

1. flowers which are purple/violet with a small white edge;

2. plant produces more than one inflorescence;

3. plants may be propagated economically and uniformly using tissue culture;

4. inflorescences are long and sturdy; and

5. relatively short, dark-green foliage.

In comparison with the parental cultivars of ‘Mamamia’, the female parent ‘Super Stupid’ has purple/red flowers and they are about 9 cm high, the male parent ‘Zuma Pixie’ has pink colored flowers and they are about 5 cm high, whereas the flowers of ‘Mamamia’ are purple/violet with a small white edge and are about 4.5 cm high.

Presently, there is no other commercial cultivar to which ‘Mamamia’ can be meaningfully compared.

### BRIEF DESCRIPTION OF THE PHOTOGRAPHS

The accompanying photographs illustrate the overall appearance of the new Phalaenopsis ‘Mamamia’ showing the colors as true as is reasonably possible with colored reproductions of this type. Colors in the photographs may differ slightly from the color values cited in the detailed botanical description, which accurately describe the color of ‘Mamamia’.

FIG. 1 shows a side view perspective of a typical flowering plant of ‘Mamamia’ in a 12 cm pot, at 16 months of age.

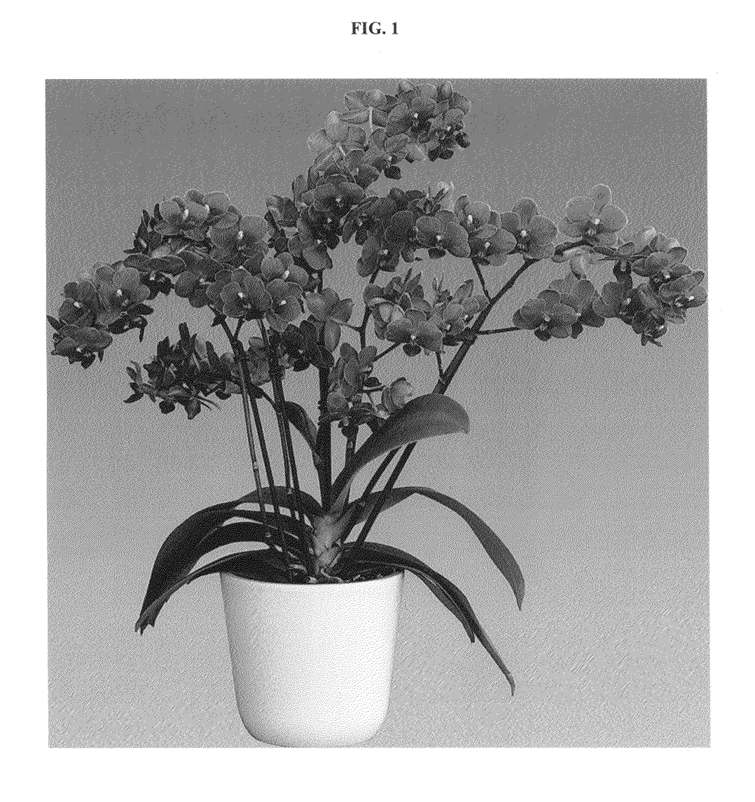

FIG. 2 shows a close-up view of the typical buds and flowers of ‘Mamamia’.

FIG. 3 shows a close-up view of the typical leaves of ‘Mamamia’.

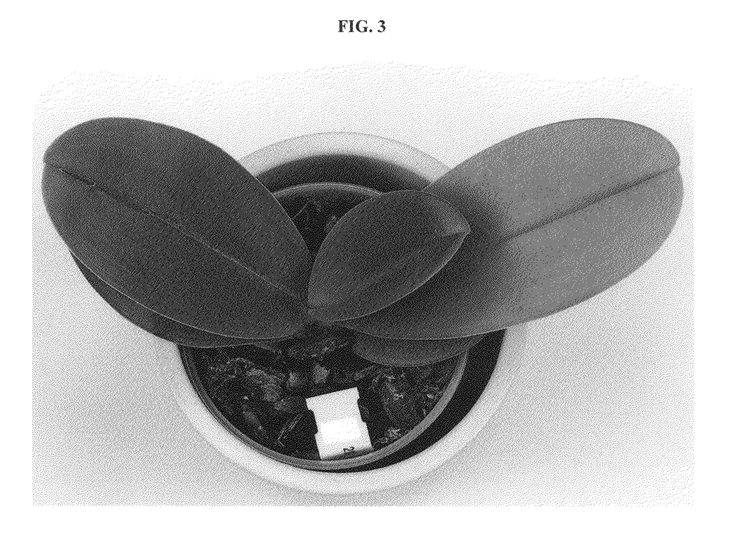

### DETAILED BOTANICAL DESCRIPTION

The new Phalaenopsis cultivar ‘Mamamia’ has not been observed under all possible environmental conditions. The phenotype of the new cultivar may vary with variations in environment such as temperature, light intensity, and day length without any change in the genotype of the plant.

The aforementioned photographs, together with the following observations, measurements and values describe plants of ‘Mamamia’ as grown in a greenhouse in Strengweg, Heemskerk, The Netherlands, under conditions which closely approximate those generally used in commercial practice. Initially, the ideal temperature to grow plants of ‘Mamamia’ is 27° C. during the day and at night. Then, during the flowering phase of ‘Mamamia’, the ideal growing temperature is 20-22° C. during the day and 18° C. at night. Light levels for growing ‘Mamamia’ are a minimum of 5,000 lux and a maximum of 10,000 lux. A balanced fertilizer with level of 200 ppm N, 87 ppm P, 168 ppm K is applied. Duration of growth of ‘Mamamia’ from potting size is between 10 and 14 months.

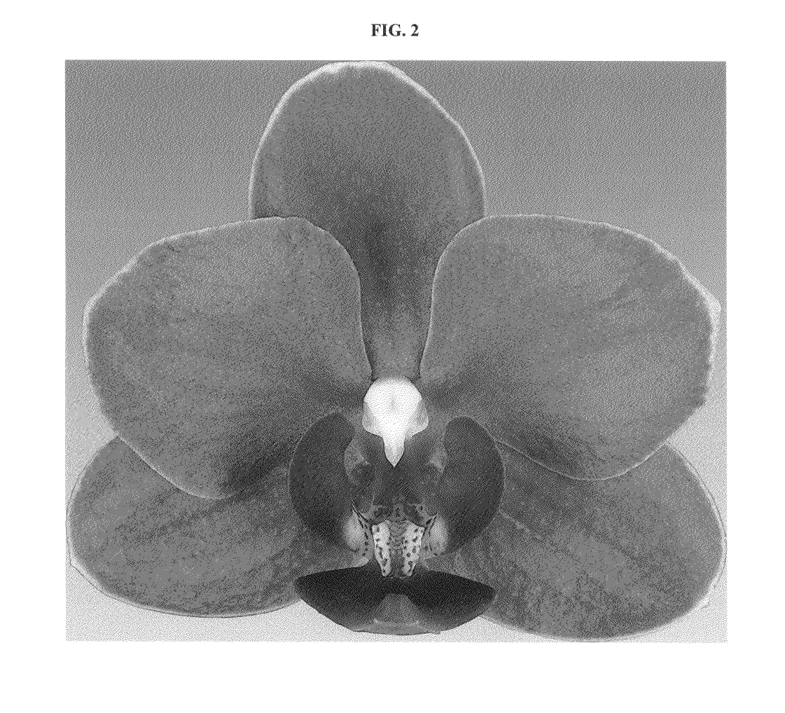

Color references are made to The Royal Horticultural Society Colour Chart (R.H.S.), 2007 edition, except where general colors of ordinary significance are used. Color values were taken under daylight conditions at approximately noon in Zaandammerweg, Assendelft, The Netherlands. The age of the ‘Mamamia’ plants described is 12 months after potting.
Classification:
   Botanical.—Phalaenopsis hybrid.
Parentage:
   Female or seed parent.—Phalaenopsis cultivar designated ‘Super Stupid’, unpatented.
   Male or pollen parent.—Phalaenopsis cultivar designated ‘Zuma Pixie’, unpatented.
Propagation:
   Type.—tissue culture.
   Rooting habit and description.—Fleshy; approximately 3 mm-6 mm wide and green in color (RHS 143A); freely branching. It takes 12 weeks for plants growing in tissue culture to initiate roots.
Plant:
   Size at maturity.—Height: about 45 cm Spread: about 45 cm.
   Growth habit.—small; dark-green leaves and a relatively normal raceme.
   Vigor.—moderate.
   Crop time.—Following asexual propagation, at about 26 weeks 2 leaves appear; at about 30 weeks 3-4 leaves appear; after a cold treatment of about 4-8 weeks at a temperature of about 19° C. about 2 peduncles with flowers appear.
Foliage:
   Quantity per plant.—About 6 to 8 leaves are produced before flowering.
   Arrangement and attachment.—half up/horizontal and on two sides.
   Overall shape of leaf.—oval, the tip is blunt and asymmetric.
   Texture (upper & underside).—smooth and leathery.
   Pubescence.—3 to 4 pairs of leaves.
   Mature leaf length.—about 14 to 22 cm.
   Mature leaf width.—about 63 to 80 mm.
   Mature leaf thickness.—about 2 mm.
   Mature leaf color.—upper side: green (RHS 137C) Under side: green (RHS 143C).
   Leaf base.—acute.
   Margin.—entire.
   Venation.—Pattern: parallel Color of midvein: upper side: green (RHS 137C) under side: green (RHS 144C).
Raceme:
   Quantity per plant.—about 2 to 5.
   Number of flowers per raceme.—about 10 to 25.
   Length.—about 35 to 45 cm.
   Diameter.—about 3 to 5 mm.
   Strength.—strong.
   Aspect.—upright.
   Texture.—glabrous and smooth.
   Color.—green (RHS 143C) with some darker green (RHS 137A).
   Internode.—Length: about 20 to 28 mm.
Inflorescence description:
   Appearance.—upright to slightly pendant, racemose inflorescence with bilaterally symmetrical flowers that open in succession beginning with the lowermost flower.
   Buds.—Height (from base to tip): about 16 mm Diameter (at midpoint): about 15 mm Shape: egg-shaped Color: green RHS 142D and purple (RHS 77B).
   Flowering time.—For an untreated plant (flowering plant that has not undergone cold-treatment where the plant grows at a temperature of 18° C. to 19° C. for about 4 to 8 weeks after a period of about 30 weeks at a temperature of 25° C.), 2 racemes appear with about 16 to 20 flower buds and flowers per inflorescence. First flowers can be expected approximately 4 to 6 months after planting a plant with a leaf diameter of 3 to 5 cm. Flowers persistent.
   Flowering longevity.—On the plant: about 4 to 6 months; lastingness of cut flowers: has not been observed.
   Fragrance.—no fragrance.
   Flower.—Rate of opening: Flowers fully opened about 2 to 3 days after petal and sepal separation. Orientation at opening: slanted upward and outward. Shape: see FIG. 2 Size: Height: about 40 mm to 50 mm Diameter: about 45 mm to 52 mm Depth of tube: about 6 mm.
   Petals.—Quantity and arrangement: six petals that are trimerous, overlapping and arranged in 2 whorls. Petals are more pronounced than sepals Arrangement: Inner whorl of petals comprises 3 petals, 2 lateral petals and labellum. 2 lateral petals: Overall shape: broadly ovate, little triangular and weakly cupped Apex: oval/round, little pointy Margin: entire and weakly undulate Base: broadly ovate Length: about 22 mm Width: about 22 mm Texture: Upper surface: smooth and satiny Under surface: smooth and satiny Color (when fully opened): upper side: main color is purple/violet (RHS N80A) with white edges (RHS NN155C) which are thin at the base and thicker at the point. Under side: main color is purple/violet (RHS N8OB and RHS N80A) with white edges (RHS NN155C). Labellum: Overall shape: 3-lobed with 2 prominent callosities at central junction of the lateral lobes and base of the midlobe. Lateral lobes of labellum fold upward about the column; the midlobe extends forward and is terminated by 2 short filiform appendages at the apex. Lateral lobes of the labellum are ovate in shape while the midlobe is triangular with a bump and a rib on it Margin: entire and weakly undulate Apex: oval Length: about 18 mm Width (not flattened): about 15 mm Texture: Upper and under surface: smooth and satiny Color (when fully opened): Midlobe: upper surface: at the base little bit white (RHS NN155C), than purple (RHS N79B) and at the end purple/violet (RHS N80A). Front edge, close to the chirri) very thin white (RHS NN155C). Under surface: main color is purple/violet (RHS N81A). Close to the edges purple (RHS N79B). Front edge, close to the chirri) very thin white (RHS NN155C). Lateral lobes: upper surface: main color is purple (RHS N79B), at the base yellow (RHS 9A) with some white (RHS NN155B). Under surface: at the base white (RHS NN155B) which runs into purple (RHS N79B). At this transition the color is grey/purple (RHS N187C). Chirri: small (about 0.5 mm) color: white (RHS NN155C) Pestle (Callosities): Length: about 4 mm Width (not flattened): about 3 mm Color: main color is white (RHS NN155B), on top some yellow (RHS 9A). Spots are red/purple (RHS 59B).
   Sepals.—Arrangement: Outer whorl comprises 3 sepals. Overall shape: elliptical and weakly cupped Margin: entire and weakly undulate Length: about 26 mm Width: about 17 mm Apex: oval/round Texture: Upper and under surface: smooth and satiny Color (when fully opened): upper surface: main color is purple/violet (RHS N80A) with small white edge (RHS NN155C). At the base lateral sepals there is some white (RHS NN155B). Under surface: edges are white (RHS NN155C) which runs into purple/violet (RHS N80B) with in the center violet (RHS 85D).
Reproductive organs:
   Arrangement.—The stamens, style and stigmas are fused into a single, short structure called the column, possessing one terminal anther with pollen grains united into a pollinia, which are covered by an anther cap. The stigma is located under the column behind the pollinia. The ovary is inferior with three carpels present. The plant has not produced seed.
   Column.—Length: about 9 mm Diameter: about 4 mm Color: purple/violet (RHS N80A).
   Pollinia.—Quantity: two Diameter: about 1 mm Color: orange (RHS 23A).
   Ovary.—Length: about 3 mm Diameter: about 4 mm Color: white (RHS N155B).
   Pedicel.—Length: about 24 mm Diameter: about 3 mm Texture: glabrous and smooth Color: from purple (RHS N77B) into red/purple (RHS 73D) with some RHS 157C.
Disease/pest resistance/susceptibility: No specific resistance or susceptibility observed
Temperature tolerance: Tolerant to a low temperature of about 15° C. and to a high temperature about 30° C.

What is claimed is:

1. A new and distinct Phalaenopsis plant named ‘Mamamia’, as illustrated and described herein.
